(19) United States
(12) Patent Application Publication
(10) Pub. No.: US 2005/0107359 A1
(43) Pub. Date: May 19, 2005

(54) Crystalline polymorphic and amorphous forms of benazepril hydrochloride

(76) Inventors: Paul Adriaan Van Der Schaaf, Allschwil (CH); Claudia Marcolli, Zurich (CH); Fritz Blatter, Reinach (CH); Martin Szelagiewicz, Munchenstein (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/500,910

(22) PCT Filed: Jul. 17, 2003
(86) PCT No.: PCT/EP03/07771

(30) Foreign Application Priority Data
Jul. 26, 2002 (EP) 02405653.3

(51) Int. Cl. A61K 31/55

(52) U.S. Cl. 514/212.07; 540/523

(57) ABSTRACT

The present invention is directed to the polymorphic Form B and the amorphous form of Benazepril hydrochloride. The present invention is also directed to processes for the preparation of Form B and the amorphous form of Benazepril hydrochloride, as well as novel processes for the preparation of Form A. Furthermore, the present invention is directed to pharmaceutical compositions comprising these crystalline forms.

The present invention is directed to a new crystalline form of Benazepril hydrochloride, an amorphous form of Benazepril hydrochloride, processes for the preparation thereof and pharmaceutical compositions comprising these forms.

The present invention relates to a new crystalline form of Benazepril hydrochloride. Benazepril hydrochloride is known by the chemical name: 3-[[(1 S)-1-(ethoxy-carbonyl)-3-phenylpropyl]amino]-2,3,4,5-tetrahydro-2-oxo-1H-1-benzazepine-1-acetic acid monohydrochloride. Benazepril has the following formula:

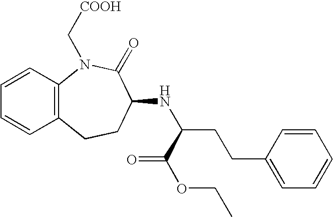

Benazepril is an orally-active ACE-inhibitor, marketed as an antihypertensive. Processes for the preparation of enantiomeric pure Benazepril hydrochloride are described in EP-A-072 352 and U.S. Pat. No. 4,575,503 and in the publications by J. W. H. Watthey et al. in J. Med. Chem. (1985), vol. 28, pages 1511-1516, and S. K. Boyer et al. in Helvetica Chimica Acta (1988), vol. 71, pages 337-342.

The processes described in the publications mentioned above result in the isolation of Benazepril hydrochloride in one defined crystalline form, herein designated as Form A. However, it is known that pharmaceutical substances can exhibit polymorphism. Polymorphism is commonly defined as the ability of any substance to have two or more different crystal structures. Drug substances may also encapsulate solvent molecules when crystallized. These solvates or hydrates are referred to as pseudopolymorphs. It is also possible that the amorphous form is encountered. Different polymorphs, pseudopolymorphs or the amorphous form differ in their physical properties such as melting point, solubility etc. These can appreciably influence pharmaceutical properties such as dissolution rate and bioavailability. It is also economically desirable that the product is stable for extended periods of time without the need for specialised storage conditions it is therefore important to evaluate polymorphism of drug substances. We now have surprisingly found a novel crystalline form of Benazepril hydrochloride, herein designated as form B, with improved stability as well as the amorphous form of Benazepril hydrochloride.

Accordingly, the present invention is directed to the polymorphic Form B, the amorphous form of Benazepril hydrochloride, processes for the preparation of Form B and the amorphous form of Benazepril hydrochloride, as well as novel processes for the preparation of Form A.

One object of the present invention is a crystalline polymorph of 3-[[(1S)-1-(ethoxy-carbonyl)-3-phenylpropyl]amino]-2,3,4,5-tetrahydro-2-oxo-1H-1-benzazepine-1-acetic acid monohydrochloride which exhibits a characteristic X-ray powder diffraction pattern with characteristic peaks expressed in d-values (Å) at 13.2 (vs), 10.7 (s), 8.8 (m), 6.4 (m), 5.87 (s), 5.75 (m), 5.35 (m), 5.26 (m), 4.87 (m), 4.66 (s), 4.40 (m), 3.86 (m), 3.79 (m), 3.66 (m), 3.60 (m), 3.57 (m), 3.52 (m), 3.45 (m), 3.40 (m), 3.36 (m), 3.27 (m), 3.18 (m), 2.95 (m), 2.72 (m), 2.65 (m);

herein designated as Form B. Here and in the following the abbreviations in brackets mean: (vs)=very strong intensity; (s)=strong intensity; (m)=medium intensity; (w)=weak intensity; and (vw)=very weak intensity.

Small changes in the experimental details can cause small deviation in the d-values of characteristic peaks in the X-ray powder diffraction patterns, see FIGS. 2 and 3 which are X-ray powder diffraction patterns for Form B.

A discussion of the theory of X-ray powder diffraction patterns can be found in “X-ray diffraction procedures” by H. P. Klug and L. E. Alexander, J. Wiley, New York (1974).

Furthermore, the present invention is directed to processes for the preparation of Form B of Benazepril hydrochloride.

Form B can generally be prepared by addition of an aqueous solution of hydrochloride (HCl) to a solution of the free base of Benazepril in an organic solvent. Examples of such organic solvents are ketones, for example acetone or methyl ethyl ketone; acetates, for example ethylacetate or isopropylacetate; nitrites, for example acetonitrile; alcohols, for example isopropylalcohol; or ethers, for example methyl-tertbutyl ether or THF. Preferred as organic solvents are C₃-C₁₀ketones, C₃-C₁₀acetates, C₂-C₁₀nitriles, C₁-C₁₀alcohols or C₂-C₁₀ethers, especially C₃-C₁₀ketones, C₃-C₁₀acetates or C₂-C₁₀ethers. Highly preferred is ethyl acetate. The weight ratio of the organic solvent to the aqueous solution of HCl is preferably 1:1 to 500:1, especially 1:1 to 100:1.Highly preferred is a weight ratio of 5:1 to 100:1. The process can, for example, be carried out at temperatures of from 10 to 60° C. Preferably, the process is carried out at ambient temperature. If desired, during the preparation process seeding with Form B can be carried out Form B can be isolated by filtration and dried in air or in vacuum.

Form B can also be prepared by stirring a suspension of Form A or the amorphous form of Benazepril hydrochloride in an organic solvent. Examples of such organic solvents are ketones, acetates, nitriles, alcohols or ethers. For these organic solvents the preferences given above apply. Highly preferred are tert-butyl methyl ether, acetone, tetrahydrofuran. The process can, for example, be carried out at temperatures of from 10 to 60° C. Form B can be isolated by filtration and dried in air or in vacuum. It is preferred that the organic solvent contains small amounts of water. The amount of water is preferably about 0.1 to 15%, most preferably about 0.5 to 10%, especially about 1 to 5% by volume of the suspension. If desired, during the preparation process seeding with Form B can be carried out.

Form B can also be prepared by stirring a suspension of Form A or the amorphous form in water. Form B can be isolated by filtration and dried in air or in vacuum. If desired, during the preparation process seeding with Form B can be carried out.

Another object of the present invention are the amorphous form of Benazepril hydrochloride and processes for the preparation thereof.

The amorphous form of Benazepril hydrochloride is characterised by a powder X-ray diffraction pattern substantially as depicted in FIG. 4.

The amorphous form of Benazepril hydrochloride can generally be prepared by evaporation of a solution of Benazepril hydrochloride in an organic solvent or water. Preferably by evaporation of a solution of Benazepril hydrochloride in one of the above organic solvents, especially in a C₂-C₁₀ketone, like acetone. According to another preferred embodiment evaporation of a solution of Benazepril hydrochloride in water is carried out. The evaporation is preferably carried out in vacuum at ambient temperature. It is also possible, to carry out evaporation at elevated temperatures.

Furthermore, the present invention is directed to processes for the preparation of Form A of Benazepril hydrochloride.

Form A can generally be prepared by mixing of a solution of Benazepril hydrochloride (preferably a concentrated solution of Benazepril hydrochloride) in an organic solvent, like C₁-C₁₀alcohols, N-methylpyrrolidone (NMP) or N,N-dimethylformamide (DMF), with a non-solvent like alkanes or acetates, especially C₄-C₁₂alkanes or C₁-C₁₀acetates, especially hexane or ethyl acetate. Preferred organic solvents are C₁-C₄alcohols, like methanol and preferably ethanol. It is preferred to add an alcoholic solution of Benazepril hydrochloride to the non-solvent, especially to heptane or ethyl acetate. If desired, during the preparation process seeding with Form A can be carried out.

Form A is preferably prepared in a waterfree medium.

Another object of the present invention are pharmaceutical compositions comprising an effective amount of crystalline polymorphic B, or the amorphous form, and a pharmaceutically acceptable carrier.

The polymorphic Form B may be used as single component or as mixtures with Form A or the amorphous form.

As to the novel polymorphic form of Benazepril hydrochloride it is preferred that these contain 25-100% by weight, especially 50-100% by weight of the novel form, based on the total amount of Benazepril hydrochloride. Preferably, such an amount of the novel polymorphic form of Benazepril hydrochloride is 75-100% by weight, especially 90-100% by weight. Highly preferred is an amount of 95-100% by weight.

The compositions of the invention include powders, granulates, aggregates and other solid compositions comprising crystalline polymorphic B or the amorphous form. In addition, the compositions that are contemplated by the present invention may further include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl, cellulose salts and other substituted and unsubstituted celluloses; starch; pregelatinized starch; inorganic diluents like calcium carbonate and calcium diphosphate and other diluents known to the pharmaceutical industry. Yet other suitable diluents include waxes, sugars and sugar alcohols like mannitol and sorbitol, acrylate polymers and copolymers, as well as pectin, dextrin and gelatin.

Further excipients that are within the contemplation of the present invention include binders, such as acacia gum, pregelatinized starch, sodium alginate, glucose and other binders used in wet and dry granulation and direct compression tableting processes. Excipients that also may be present in the solid compositions further include disintegrants like sodium starch glycolate, crospovidone, low-substituted hydroxypropyl cellulose and others. In addition, excipients may include tableting lubricants like magnesium and calcium stearate and sodium stearyl fumarate; flavorings; sweeteners; preservatives; pharmaceutically acceptable dyes and glidants such as silicon dioxide.

The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of Benazepril hydrochloride whereupon the properties that distinguish the solid forms of Benazepril hydrochloride are lost. However, the use of the novel forms to prepare such solutions is considered to be within the contemplation of the invention.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and N desired, they may be employed with suitable plasticizers and/or extending agents. A coated tablet may have a coating on the surface of the tablet or may be a tablet comprising a powder or granules with an enteric-coating.

Preferred unit dosages of the pharmaceutical compositions of this invention typically contain from 0.5 to 100 mg of the novel Benazepril hydrochloride forms or mixtures thereof with each other or other forms of Benazepril hydrochloride. More usually, the combined weight of the Benazepril hydrochloride forms of a unit dosage are from 2.5 mg to 80 mg, for example 5, 10, 20 or 40 mg.

The following Examples illustrate the invention in more detail. Temperatures are given in degrees Celsius.

### EXAMPLE 1

### Preparation of Polymorphic Form B

100 mg of Benazepril hydrochloride Form A was suspended in a mixture of 2 ml tert-butyl methyl ether and 0.1 ml water. This suspension was stirred for 14 hours at 20° C. 78 mg of Benazepril hydrochloride Form B was obtained after filtration and dried in vacuum at 30° C. The obtained Form B was characterized by X-ray powder diffraction, see FIG. 2.

### EXAMPLE 2

### Preparation of Polymorphic Form B

161 mg Benazepril hydrochloride Form A was suspended in 3 ml acetone and stirred for 20 hours at 20° C. This suspension was filtered and dried in air at 30° C. X-ray powder diffraction showed the product to be polymorphic Form B, see FIG. 3.

### EXAMPLE 3

### Preparation of Polymorphic Form B

160 mg Benazepril hydrochloride Form A was suspended in 2 ml THF. This suspension was stirred at ambient temperatures for 5 hours. This suspension was filtered and dried in air at 30° C. X-ray powder diffraction showed the product to be polymorphic Form B.

### EXAMPLE 4

### Preparation of Polymorphic Form B

86 mg Benazepril free base was dissolved in 3 ml ethyl acetate. Then 0.1 ml of an aqueous 2 molar solution of HCl was added. After adding an additional 3 ml of ethyl acetate and stirring for 3 hours, the product was obtained by filtration and dried in air at ambient temperature. X-ray powder diffraction showed the product to be polymorphic Form B.

### EXAMPLE 5

### Preparation of Polymorphic Form A

Reference example: 2.4 gram Benazepril free base was dissolved in 60 ml diethyl ether. This solution was stirred for 20 minutes under a gentle stream of HCl gas. The white suspension was stirred for an additional 15 minutes and then filtered. The white solid was dried in vacuum at 40° C. (35 mbar). The product (2.3 gram) was characterized by X-ray powder diffraction, see FIG. 1.

### EXAMPLE 6

### Preparation of Polymorphic Form A

111 mg Benazepril hydrochloride was dissolved in 0.8 ml water-free ethanol. This solution was rapidly added to 10 ml heptane at 20° C. While stirring, the suspension was slowly cooled to 5° C. Then the white precipitate was filtered and dried in vacuum. X-ray powder diffraction showed the product to be polymorphic Form A.

### EXAMPLE 7

### Preparation of the Amorphous Form

100 mg Benazepril hydrochloride was dissolved in 2 ml water. The solution was filtered and the obtained clear solution was evaporated to dryness at 50° C. in vacuum (300 mbar). The obtained white powder was characterized by DSC (Tg=76° C.) and X-ray powder diffraction, see FIG. 4.

### BRIEF DESCRIPTION OF THE DRAWINGS

FIG. 1 is a characteristic X-ray powder diffraction pattern for Form A.

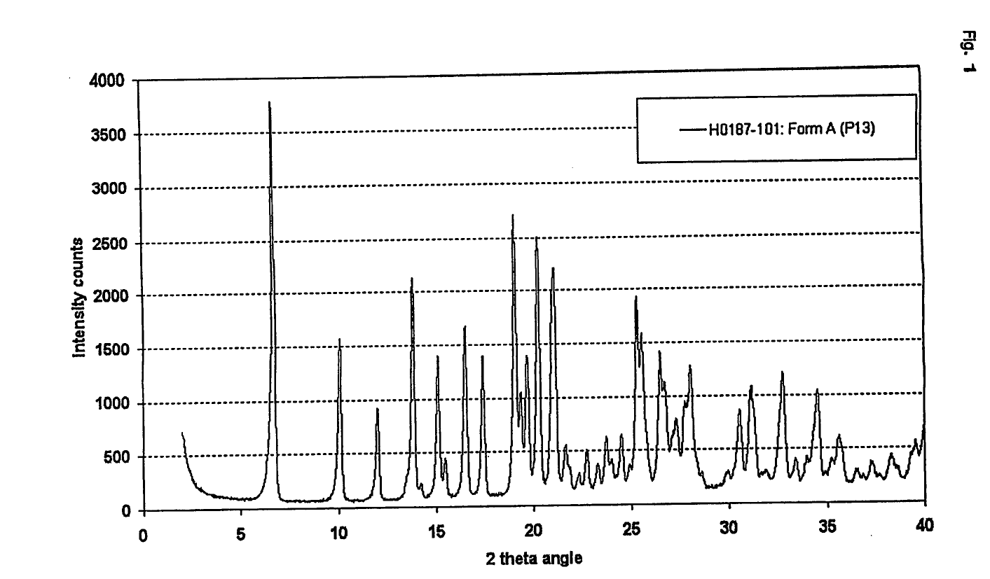

FIG. 2 is a characteristic X-ray powder diffraction pattern for Form B.

FIG. 3 is another characteristic X-ray powder diffraction pattern for Form B.

FIG. 4 is a characteristic X-ray powder diffraction pattern for the amorphous form.

1. A crystalline polymorph B of 3-[[(1S)-1-(ethoxy-carbonyl)-3-phenylpropyl]amino]-2,3,4,5-tetrahydro-2-oxo-1H-1-benzazepine-1-acetic acid monohydrochloride which exhibits a characteristic X-ray powder diffraction pattern with characteristic peaks expressed in d-values (Å) at 13.2 (vs), 10.7 (s), 8.8 (m), 6.4 (m), 5.87 (s), 5.75 (m), 5.35 (m), 5.26 (m), 4.87 (m), 4.66 (s), 4.40 (m), 3.86 (m), 3.79 (m), 3.66 (m), 3.60 (m), 3.57 (m), 3.52 (m), 3.45 (m), 3.40 (m), 3.36 (m), 3.27 (m), 3.18 (m), 2.95 (m), 2.72 (m), 2.65 (m); wherein (vs)=very strong intensity; (s)=strong intensity; (m)=medium intensity.

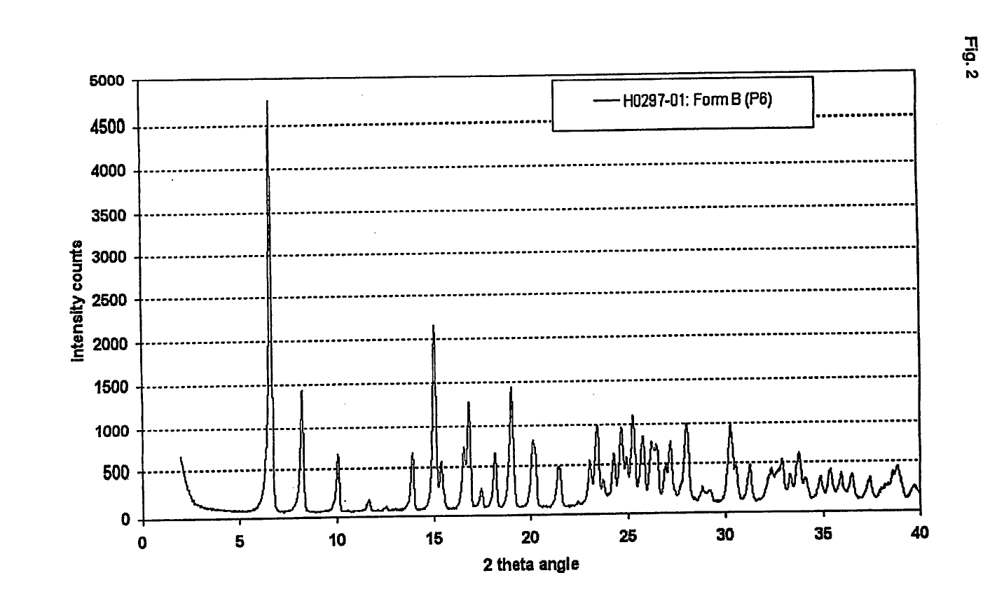

2. A crystalline polymorph B of 3-[[(1S)-1-(ethoxy-carbonyl)-3-phenylpropyl]amino]-2,3,4,5-tetrahydro-2-oxo-1H-1-benzazepine-1-acetic acid monohydrochloride having an X-ray powder diffraction pattern substantially as depicted in FIG. 2.

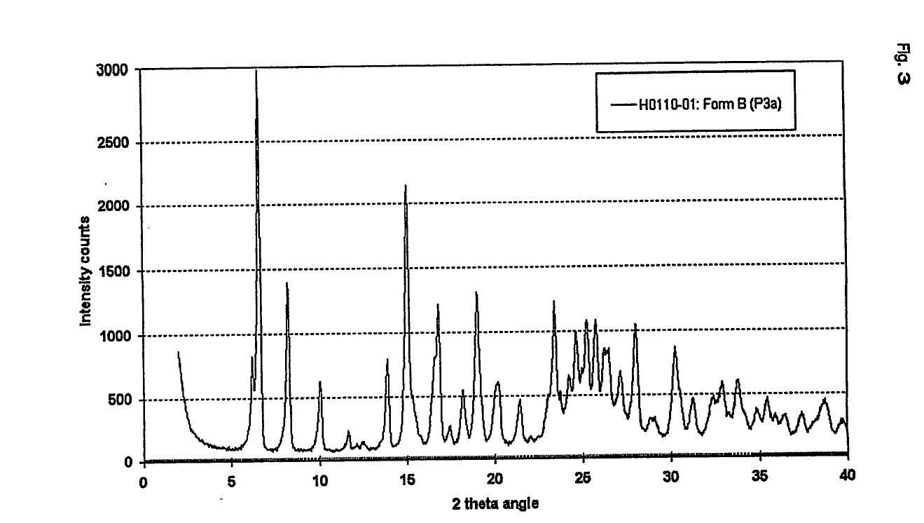

3. A crystalline polymorph B of 3-[[(1S)-1-(ethoxy-carbonyl)-3-phenylpropyl]amino]-2,3,4,5-tetrahydro-2-oxo-1H-1-benzazepine-1-acetic acid monohydrochloride having an X-ray powder diffraction pattern substantially as depicted in FIG. 3.

4. An amorphous form of 3-[[(1S)-1-(ethoxy-carbonyl)-3-phenylpropyl]amino]-2,3,4,5-tetrahydro-2-oxo-1H-1-benzazepine-1-acetic acid monohydrochloride.

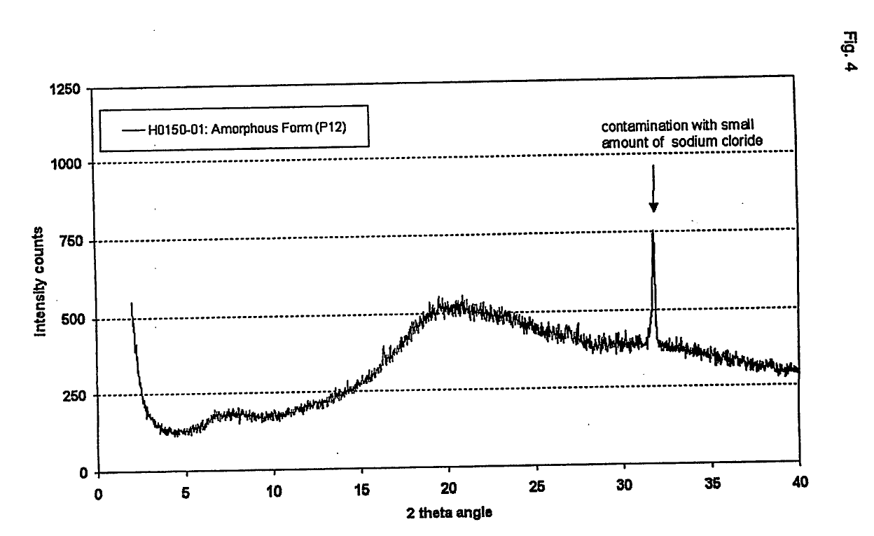

5. An amorphous form of 3-[[(1S)-1-(ethoxy-carbonyl)-3-phenylpropyl]amino]-2,3,4,5-tetrahydro-2-oxo-1H-1-benzazepine-1-acetic acid monohydrochloride according to claim 4 having a powder X-ray diffraction pattern substantially as depicted in FIG. 4.

6. A process for the preparation of a crystalline polymorph according to claim 1, wherein an aqueous solution of hydrochloride is added to a solution of the free base 3-[[(1S)-1-(ethoxy-carbonyl)-3-phenylpropyl]amino]-2,3,4,5-tetrahydro-2-oxo-1H-1-benzazepine-1-acetic acid monohydrochloride in an organic solvent.

7. A process according to claim 6, wherein the organic solvent is a C₃-C₁₀ketone, C₃-C₁₀acetate, C₂-C₁₀nitrile, C₁-C₁₀alcohol or C₂-C₁₀ether, or a mixture thereof.

8. A process for the preparation of a crystalline polymorph according to claim 1, wherein a suspension of Form A or the amorphous form 3-[[(1S)-1-(ethoxy-carbonyl)-3-phenylpropyl]amino]-2,3,4,5-tetrahydro-2-oxo-1H-1-benzazepine-1-acetic acid monohydrochloride is stirred in an organic solvent.

9. A process according to claim 8, wherein the organic solvent is a C₃-C₁₀ketone, C₃-C₁₀acetate, C₂-C₁₀nitrile, C₁-C₁₀alcohol or C₂-C₁₀ether, or a mixture thereof.

10. A process according to claim 8, wherein the organic solvent is selected from the group consisting of acetone, 1-butanol, 2-butanol, butyl actetate, tert-butylmethyl ether, cumene, dimetylsulfoxide, ethanol, ethylether, ethylformiate, heptane isobutylacetate, isopropyl acetate, methylacetate 3-methyl-1-butanol and methylethyl ketone.

11. A process according to claim 8, wherein the organic solvent is selected from the group consisting of acetone, methyl ethyl ketone; ethylacetate, isopropylacetate, acetonitrile, isopropylalcohol, methyl-tertbutyl ether and THF.

12. A process according to claim 8, wherein the organic solvent contains small amounts of water.

13. A process according to claim 12, wherein the amount of water is 0.1 to 15% by volume of the suspension of 3-[[(1S)-1-(ethoxy-carbonyl)-3-phenylpropyl]amino]-2,3,4,5-tetrahydro-2-oxo-1H-1-benzazepine-1-acetic acid monohydrochloride.

14. A process according to claim 13, wherein the amount of water is 0.5 to 10% by volume of the suspension of 3-[[(1S)-1-(ethoxy-carbonyl)-3-phenylpropyl]amino]-2,3,4,5-tetrahydro-2-oxo-1H-1-benzazepine-1-acetic acid monohydrochloride.

15. A process for the preparation of a crystalline polymorph according to claim 1 wherein a suspension of Form A or the amorphous form of 3-[[(1S)-1-(ethoxy-carbonyl)-3-phenylpropyl]amino]-2,3,4,5-tetrahydro-2-oxo-1H-1-benzazepine-1-acetic acid monohydrochloride is stirred in water.

16. A process according to claim 15, wherein 3-[[(1S)-1-(ethoxy-carbonyl)-3-phenylpropyl]amino]-2,3,4,5-tetrahydro-2-oxo-1H-1-benzazepine-1-acetic acid monohydrochloride is isolated by filtration and dried in air or vacuum.

17. A process for the preparation of a crystalline polymorph according to claim 1, wherein seeding is carried out with crystals of the crystalline polymorph according to claim 1.

18. A process for the preparation of the amorphous form according claim 4, wherein a solution 3-[[(1S)-1-(ethoxy-carbonyl)-3-phenylpropyl]amino]-2,3,4,5-tetrahydro-2-oxo-1H-1-benzazepine-1-acetic acid monohydrochloride in an organic solvent or in water is evaporated to dryness.

19. A process according to claim 18, wherein the organic solvent is a C₃-C₁₀ketone.

20. A process according to claim 19, wherein the organic solvent is acetone.

21. A process for the preparation of crystalline polymorph Form A of 3-[[(1S)-1-(ethoxy-carbonyl)-3-phenylpropyl]amino]-2,3,4,5-tetrahydro-2-oxo-1H-1-benzazepine-1-acetic acid monohydrochloride, wherein a concentrated solution of 3-[[(1S)-1-(ethoxy-carbonyl)-3-phenylpropyl]amino]-2,3,4,5-tetrahydro-2-oxo-1H-1-benzazepine-1-acetic acid monohydrochloride in an organic solvent is mixed with a non-solvent.

22. A process according to claim 21, wherein the organic solvent is an C₁-C₁₀alcohol, tetrahydrofuran, N-methylpyrrolidone or N,N-dimethylformamide and the non-solvent is a C₄-C₁₂alkane or C₁-C₁₀acetate.

23. A process according to claim 22, wherein a solution of 3-[[(1S)-1-(ethoxy-carbonyl)-3-phenylpropyl]amino]-2,3,4,5-tetrahydro-2-oxo-1H-1-benzazepine-1-acetic acid monohydrochloride in a C₁-C₄alcohol is mixed with heptane.

24. A process according to claim 21, wherein seeding with crystals of the Form A of 3-[[(1S)-1-(ethoxy-carbonyl)-3-phenylpropyl]amino]-2,3,4,5-tetrahydro-2-oxo-1H-1-benzazepine-1-acetic acid monohydrochloride is carried out.

25. A pharmaceutical composition comprising an effective amount of a crystalline polymorphic form of 3-[[(1S)-1-(ethoxy-carbonyl)-3-phenylpropyl]amino]-2,3,4,5-tetrahydro-2-oxo-1H-1-benzazepine-1-acetic acid monohydrochloride according to claim 1, and a pharmaceutically acceptable carrier.

26. A process according to claim 6, wherein 3-[[(1S)-1-(ethoxy-carbonyl)-3-phenylpropyl]-amino]-2,3,4,5-tetrahydro-2-oxo-1H-1-benzazepine-1-acetic acid monohydrochloride is isolated by filtration and dried in air or vacuum.

27. A pharmaceutical composition comprising an effective amount of the amorphous form of 3-[[(1S)-1-(ethoxy-carbonyl)-3-phenylpropyl]amino]-2,3,4,5-tetrahydro-2-oxo-1H-1-benzazepine-1-acetic acid monohydrochloride according to claim 4, and a pharmaceutically acceptable carrier.
